(19) United States
(12) Patent Application Publication
(10) Pub. No.: US 2023/0061665 A1
(43) Pub. Date: Mar. 2, 2023

(54) DETERMINING SUBTYPES OF SCHIZOPHRENIA IN A SUBJECT, TREATMENT OF SCHIZOPHRENIA, MEDICAMENT FOR TREATING SCHIZOPHRENIA AND DETERMINING THE EFFICACY OF SUCH MEDICATION

(71) Applicant: NATIONAL INSTITUTE OF MENTAL HEALTH, Klecany (CZ)

(72) Inventors: Filip SPANIEL, Zdiby (CZ); Vaclav CAPEK, Borotin (CZ); Antonin SKOCH, Prague (CZ)

(73) Assignee: NATIONAL INSTITUTE OF MENTAL HEALTH, Klecany (CZ)

(21) Appl. No.: 17/793,213

(22) PCT Filed: Jan. 15, 2021
(86) PCT No.: PCT/EP2021/050873

§ 371 Date: Jul. 15, 2022

(30) Foreign Application Priority Data
Jan. 15, 2020 (GB) 2000634.2

(51) Int. Cl. A61B 5/055; A61B 5/0515; A61B 5/00

(52) U.S. Cl. CPC A61B 5/055; A61B 5/0515; A61B 5/0042; A61B 5/4064

(57) ABSTRACT

Methods of categorisation of schizophrenia sufferers into subtypes based on changes in brain morphology, together with associated blood biomarkers are provided. The methods allow for more accurate treatment and diagnosis of schizophrenia.

### INTRODUCTION

Schizophrenia has been regarded as heterogeneous disorder since the establishment of its nosological entity. The paradigm of the heterogeneity that would stem from distinct subtypes of patients with different neurobiology has not been widely accepted so far. Conceptualizations of this issue vary between extremes ranging from propositions of unitary pathophysiological process that is shared across patients to a notion of overwhelming inter-individual differences that preclude any reliable biological subtypization. The present state of knowledge, though, favours the hypothesis that schizophrenia represents rather a syndrome that incorporates several distinct illnesses. Yet, the unequivocal means to identify subtypes and predict individual prognosis remain undefined. Such an approach could, however, ultimately enhance prognostic accuracy and facilitate investigation of cause of psychosis.

There is substantial evidence for the presence of brain volume abnormalities in patients with schizophrenia, and evidence for excessive tissue loss is accumulating. Although it is now increasingly clear that neither neuronal loss and gliosis nor a clinical characteristic of true neurodegeneration occurs in schizophrenia, the evidence of dynamic neuroprogressive post-onset brain changes seem to be unequivocal.

Longitudinal neuroimaging studies have been known to be a useful tool as measurements in anatomical changes are associated with the onset of psychotic illness, as neuroimaging data may be acquired before and after psychosis occurs. Longitudinal MRI studies indicate that the underlying pathological process of the grey matter deficit appears to be especially active 2 to 3 years post-onset, before slowing down during the chronic phase (DIETSCHE, Bruno; KIRCHER, Tilo; FALKENBERG, Irina. Structural brain changes in schizophrenia at different stages of the illness: a selective review of longitudinal magnetic resonance imaging studies. Australian & New Zealand Journal of Psychiatry, 2017, 51.5: 500-508).

Previous cross-sectional studies building on brain structural alterations suggested the possibility of teasing apart different neuroanatomical patterns in schizophrenia. However, cross-sectional designs and recruitment of chronic patient populations, typical for those studies, disregard a crucial dimension: a longitudinal trajectory of structural changes during critical period of the course of the illness.

The present invention is directed to a method of quantifying longitudinal changes in cortical thickness and cortical folding to characterise major reorganisations in the grey matter in the early stage of schizophrenia spectrum disorder.

Cortical thickness (CTh) is related to neuronal structural complexity features such as neuronal size, presynaptic terminals, and complexity of dendritic arborizations. Changes in CTh could be occurring well before a decrease in neuronal viability or neuronal death takes place, suggesting a sensitivity of this metric even to preclinical stages of a neuroprogression.

Cortical folding which is also known as gyrification index (GI) represents a surface-based morphometry (SBM) metric that quantifies the ratio of inner sulcal folds compared with the outer smooth surface of the cortex. Although the folding typology has been traditionally perceived as a postnatally-stable marker for variations in early brain development, recent evidence highlights changes in cortical gyrification as a sensitive state-marker related to neuropsychiatric disease progression, disease staging in neurodegeneration, brain exposure to environmental variables, like nutrition, or temporal hormonally primed brain changes.

The data-driven methods based on longitudinal morphometry are known to provide an unbiased approach to detect atrophy subtypes in Alzheimer's and Parkinson's disease. However, prior to the present invention, the practicality of such studies in Schizophrenia, which is associated with such a wide range of clinically psychotic, negative, and cognitive symptoms, had yet to be confirmed.

In view of the foregoing, the present invention provides a method for identification of distinct neurophenotypes of schizophrenia based on longitudinal changes in the cortical mantle and sulcal anatomy.

### BRIEF DESCRIPTION OF THE FIGURES

FIG. 1 shows brain volume longitudinal differences between V1 and V2 without ventricles.

FIG. 2 provides clustering analysis of divergent longitudinal morphometry pathways in FES patients.

FIG. 3 shows clusters of cortex volume at baseline.

FIG. 4 provides clusters of cortex thickness after one year.

FIG. 5 shows the overall change in grey matter between different clusters.

FIG. 6 provides hierarchical clustering of cortical parcels within separate C1-C3 patient clusters. Negative values on (x) are indexing CTh reduction during one-year follow up, whereas positive values show cortex thickening. This density plot illustrates a more detailed complex patchwork of cortical reorganizations inside patient clusters identified in CTh model.

FIG. 7 depicts overlap of two clustering methods: (x) 3-cluster solution based on CTh change, (y) 3-cluster solution based on change in gyrification index. Overlap of the two methods is statistically significant (Fisher's exact test, p=0.001)

FIG. 8 demonstrates the results of longitudinal VBM between three clusters.

FIG. 9 shows longitudinal GM loss in clusters 1 and 2.

FIG. 10 represents DBM analysis between clusters 1 and 2.

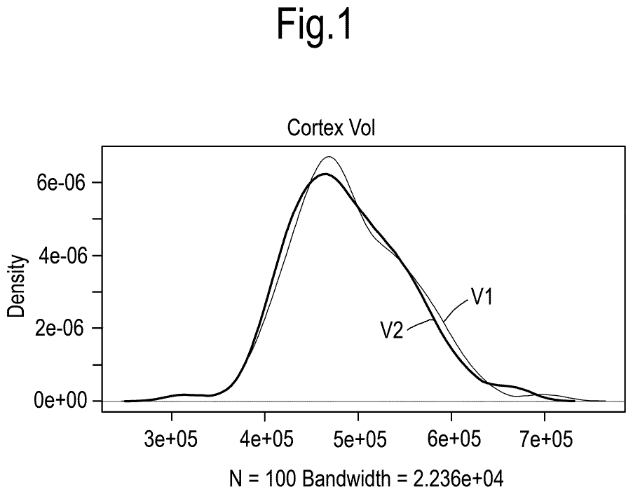

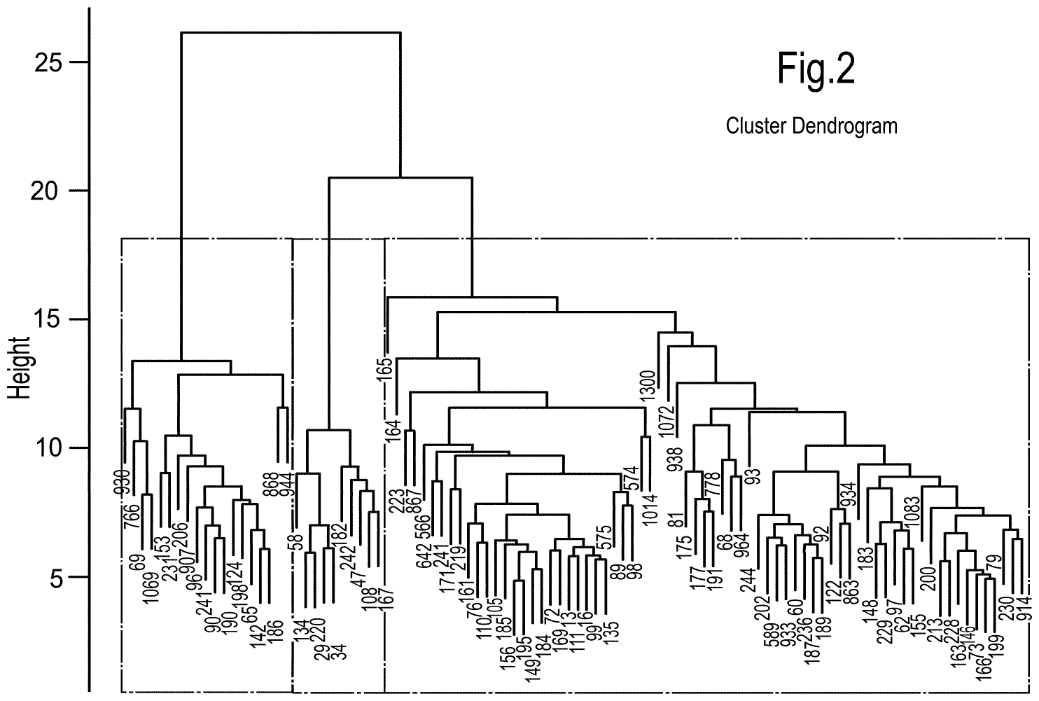

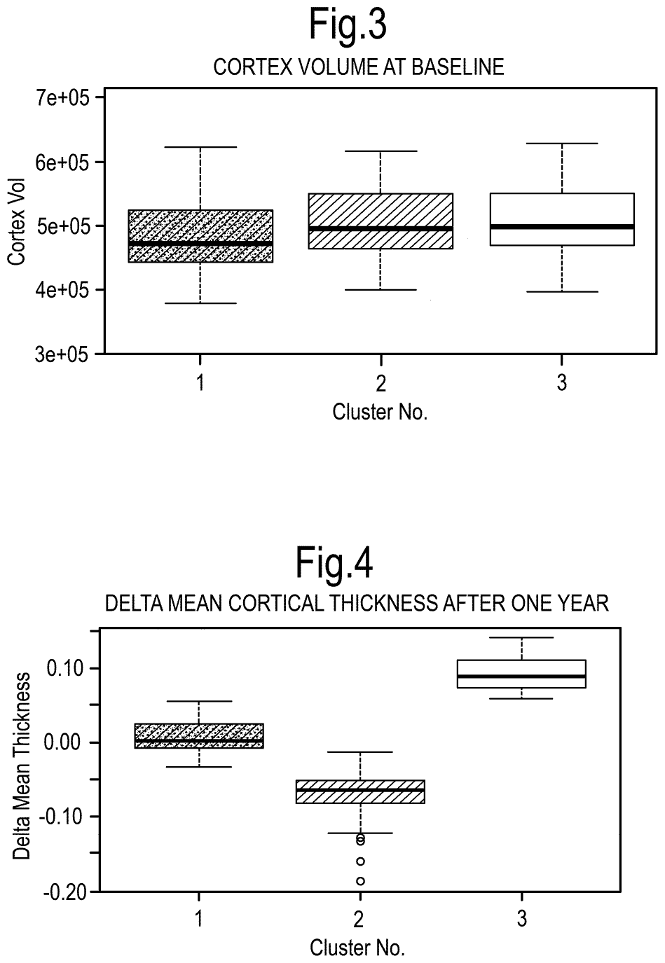

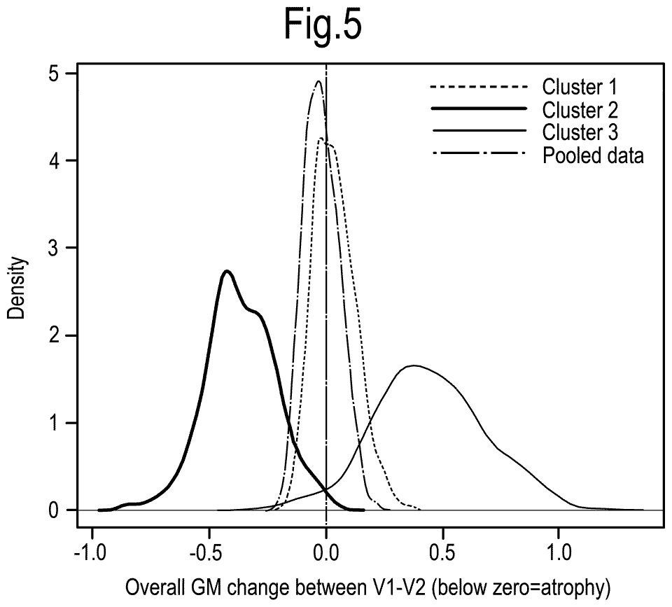

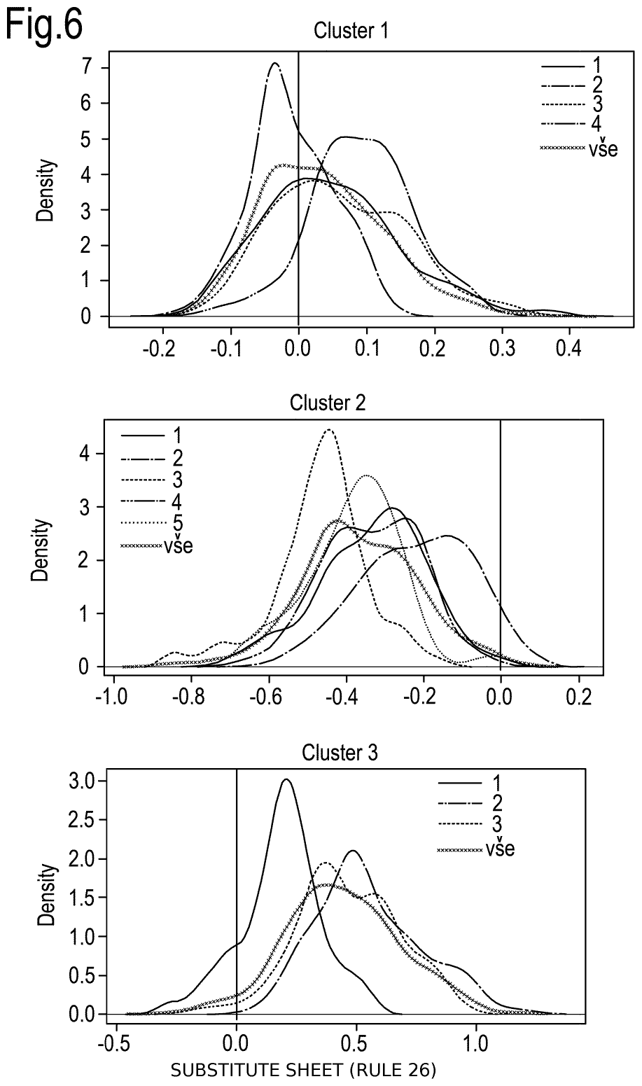

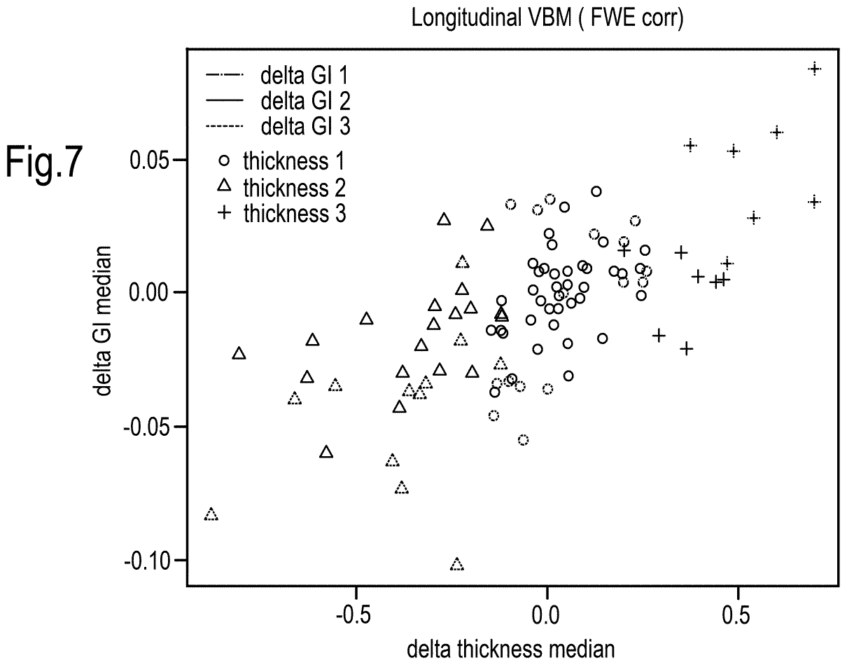

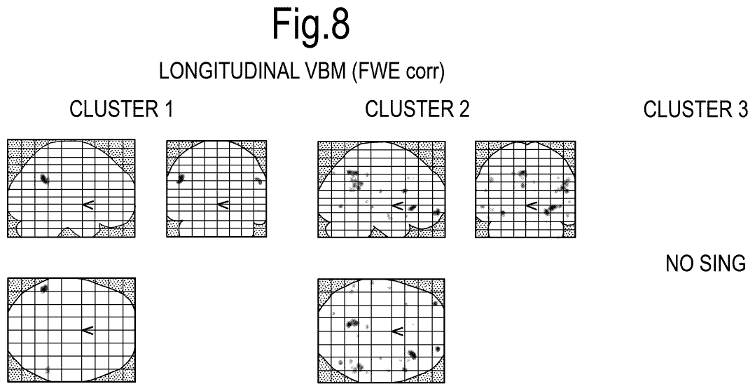

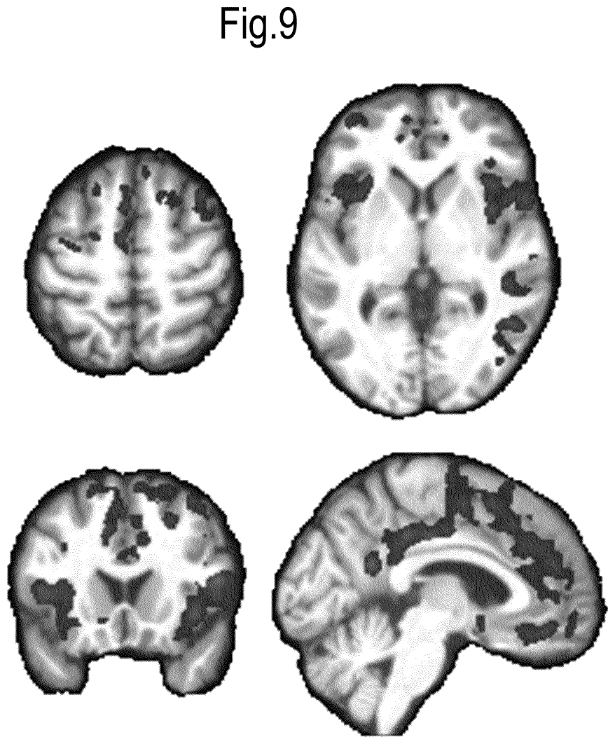

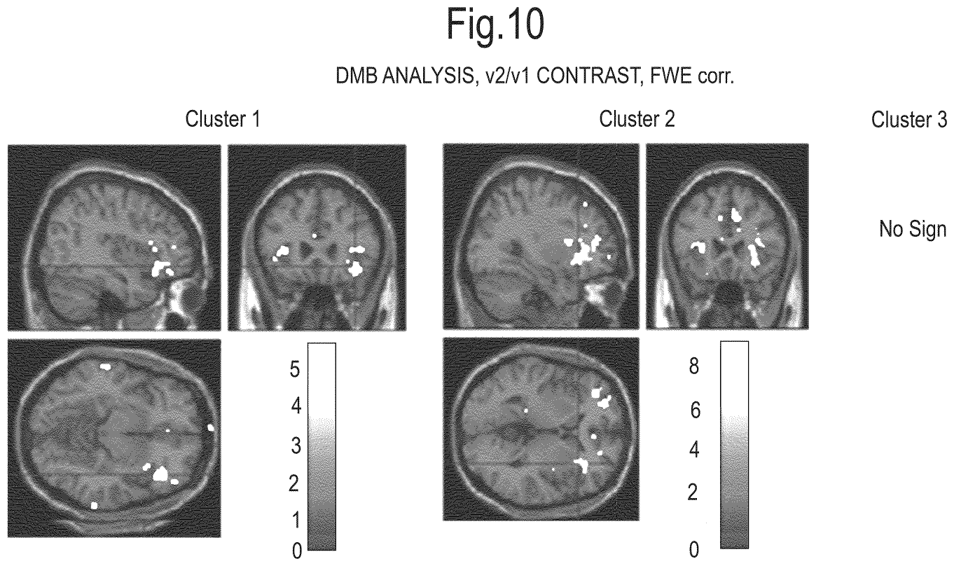

### STATEMENT OF THE INVENTION

According to a first embodiment of the invention, there is provided a method for diagnosing distinct subtypes of schizophrenia said method comprising:

obtaining first structural magnetic resonance imaging (sMRI) brain scan at a first point in time;

obtaining a second structural magnetic resonance imaging brain scan at a later point in time;

processing the first and second scans to obtain first and second measures of cortical thickness;

comparing the first and second measures to establish the change in cortical thickness;

categorising the subject as belonging to a distinct subtype of schizophrenia according to the changes in cortical thickness.

According to a second embodiment of the invention, there is provided a method for diagnosing a neurodegenerative subtype of schizophrenia by detecting reorganisation in grey matter of a subject's brain, said method comprising, obtaining first structural magnetic resonance imaging (sMRI) brain scan at a first point in time;

obtaining a second structural magnetic resonance imaging brain scan at a later point in time;

processing the first and second scans to obtain first and second measures of gyrification index;

comparing the first and second measures to establish the change in gyrification index;

categorising the subject as belonging to distinct subtypes of schizophrenia according to the changes in gyrification index.

According to a third embodiment, the invention relates an in vitro method for diagnosing distinct subtypes of schizophrenia by determining the levels of one or more biomarkers in a sample obtained from a patient at the earliest time after the onset of schizophrenia, selected from the panel consisting of S100B; NF-L; NSE; GFAP; and/or UCH-L1

According to a fourth embodiment, the invention relates to a method of treatment of schizophrenia in a patient, comprising the steps of:

i. determining the subtype of schizophrenia in the patient by a method according to the invention;

ii. selecting an antipsychotic medication based on the determination in step i; and

iii. administering the antipsychotic medication to the patient.

According to a fifth embodiment, the invention relates to an antipsychotic medicament for use in a method of treatment of a patient suffering from schizophrenia, the method comprising steps of:
i. determining the subtype of schizophrenia in the patient by a method according to the invention;
ii. selecting an antipsychotic medication based on the determination in step i; and
iii. administering the antipsychotic medication to the patient.

According to a sixth embodiment, the invention relates to a method of determining the efficacy of an antipsychotic medication in a patient suffering from schizophrenia, comprising the steps of:
i. determining the subtype of schizophrenia in the patient by a method according to the invention;
ii. administering the medication to the patient; and
iii. assessing the efficacy of the medication in reducing the symptoms of schizophrenia.

According to a seventh embodiment, the invention relates to A method of determining the efficacy of antipsychotic medication to patients suffering from a subtype of schizophrenia, comprising the steps of:
i. providing a cohort of patients suffering from schizophrenia;
ii. determining the subtype of schizophrenia patient by a method according to the invention; and
iii. administering the medication to the patients; and
iv. assessing the efficacy of the medication in alleviating the symptoms of schizophrenia in each subtype.

### DETAILED DESCRIPTION AND PREFERRED EMBODIMENTS

The present invention relates to a method for diagnosing a distinct subtypes of schizophrenia by detecting reorganisation in grey matter of a subject's brain.

Certain methods of the invention relate to measurement of the certain aspects of the brain by Structural Magnetic Resonance Imaging (sMRI). Structural MRI can be used to quantify spatial patterns of brain atrophy using a T1-weighted sequence, which discriminates well between gray and white matter. This contrast has been used in research since the mid-1980s, when magnetic resonance became a viable method for non-invasive brain imaging (Besson et al., 1985; Fazekas, Chawluk, & Alavi, 1987; Reiman & Jagust, 2012).

In one embodiment, the methods relate to obtaining a number of sMRI images at time intervals. At least two images are required, but more (e.g. three, four or more) may be obtained. The two images are separated by an interval of time. This interval is preferably six months or more; more preferably, it is one year or more. If more than two images are obtained, it is preferred that these are at regular intervals.

It is preferred that the first sMRI image is taken as soon as possible after a diagnosis of schizophrenia is made, such as within one month, preferably within one week.

Cortical thickness is a brain morphometric measure used to describe the combined thickness of the layers of the cerebral cortex in the brain, either in local terms or as a global average for the entire brain. Given that cortical thickness roughly correlates with the number of neurons within an ontogenetic column, it is often taken as indicative of the cognitive abilities of an individual, albeit the latter are known to have multiple determinants.

In the living brain, cortical thickness is commonly determined on the basis of the grey matter set in segmented neuroimaging data, usually from the local or average distance between the white matter surface and the pial surface. Typical values in adult humans are between 1.5 and 3 mm, and during aging, a decrease (also known as cortical thinning) on the order of about 10 μm per year can be observed.

In one aspect, the methods of the invention are directed to measurement of cortical thickness. Surprisingly, it has been found that schizophrenia sufferers fall into three distinct categories or clusters, namely those showing an increase in cortical thickness, those showing a decrease, and those showing little or no change.

Gyrification index is a metric that quantifies the amount of cortex buried within the sulcal folds as compared with the amount of cortex on the outer visible cortex. A cortex with extensive folding has a large gyrification index, whereas a cortex with limited folding has a small gyrification index.

In one aspect, the methods of the invention are directed to measurement of gyrification index. The change in gyrification index provides a further indicator of the subtype of schizophrenia from which a patient is suffering.

In a further aspect, the physiological changes identified in the brain morphometry are found to be associated with different profiles of blood biomarkers. Hence, this provides an in vitro method for subtyping of schizophrenia.

The previously unappreciated existence of these subtypes provides methods for more effective treatment of schizophrenia. For example, patients of each subtype respond differently to different antipsychotic medicines. Hence, the categorization of schizophrenia sufferers into clusters enables the clinician to more accurately administer the appropriate medicament.

Furthermore, the methods of categorization described herein enable clinical trials of new antipsychotic medicines to be more effectively conducted. Certain subtypes of schizophrenia described herein will respond better to drug candidates. This enables a personalised medicine approach allowing the development of therapies specific for each subtype.

Methods

A hierarchical cluster analysis (CORDES, Dietmar, et al. Hierarchical clustering to measure connectivity in fMRI resting-state data. Magnetic resonance imaging, 2002, 20.4: 305-317) was performed in first-episode schizophrenia spectrum (FES) patients using data on within-subject changes in cortical thickness and cortical folding after the onset of the disease and 12 months later. To control for the physiological effect of time, matched healthy controls (HC) were also scanned twice, 12 months apart.

As a complementary approach to hierarchical clustering, the results were validated on univariate analysis employing Voxel-based morphometry (VBM) and, Deformation-based morphometry (DBM).

First—episode schizophrenia patients have been included and structural magnetic resonance imaging (sMRI) brain scans have been gathered. Measurements of cortical thickness (CTh) were taken with submillimetre resolution. Two sMRI scans have been acquired on each participant at 1-year intervals. Patients and controls were scanned identically with the same scanner and scanning protocol. These data allowed generation of an estimate of annual CTh change at cortical parcels, in each participant. The repeat sMRI measures of brain anatomy allowed creation of person-specific maps of anatomical changes and thus clustering of patients into diverse longitudinal trajectories that exist within the reorganization of cortical mantle in the early stage of schizophrenia.

Surface-Based Morphometry

To estimate cortical thickness, images were processed with the FreeSurfer software package. In brief, pre-processing included intensity normalization, removal of non-brain tissue, transformation to Talairach-like space, segmentation of gray-white matter tissue, and tessellation and smoothing of the white matter boundary. White matter surfaces were then deformed toward the gray matter boundary at each vertex. Cortical thickness was calculated based on the distance between white and gray matter boundaries at each vertex. The entire cortex of each study subject was subsequently visually inspected, and inaccuracies in segmentation were manually edited.

Following pre-processing, the cortical surface was parcellated into multiple contiguous areas. Here, a Gordon atlas based on resting-state functional connectivity (RSFC) boundary maps were applied (GORDON, Evan M., et al. Generation and evaluation of a cortical area parcellation from resting-state correlations. Cerebral cortex, 2014, 26.1: 288-303).

Subsequently, the mean CTh (in mm) and GI values, respectively, for both baseline and one-year follow up MRI scanning were extracted from each of 333 cortical parcels (161 and 162 regions from the left and right hemispheres, respectively) covering all the cortical mantle. Specifically, for both baseline and one-year follow up MRI, parcel-averaged cortical thickness (and gyrification index) estimates were computed by averaging across all vertices comprising each region. This yielded a vector of 333 regional cortical thickness (or gyrification index) estimates for each study subject. Further, within-subject change scores in CTh or GI (baseline CTh subtracted from 1-year follow-up) were computed for all Gordon parcels separately. Those two separated variables entered hierarchical clustering analysis with Euclidean distance metric that rely on determining the latent subclasses of patients with globally similar patterns of (i) progressive changes cortical thickness (PCT) and (ii) progressive changes in gyrification index (PCGI), respectively. Hierarchical clustering methods build a succession of clusters: data-points (individual 333 vectors of CTh and GI between-visits difference values) are first grouped into clusters, and the clusters themselves are merged into groups at a second level according to their similarity, building a tree depicting the hierarchical dependence structure across data points. The decision is further illuminated by a dendrogram, showing the groups and their proximity, herein encoded as the Euclidean similarity distance (M. Forina, C. Armanino, V. Raggio Clustering with dendrograms on interpretation variables Analytica Chimica Acta, 454 (1) (2002), pp. 13-19).

Hierarchical clustering minimizes the variance of the distances from each individual in a cluster to the cluster center, thereby ensuring similarity of the individuals within a cluster. A particularly strong aspect of the method is that it enables identifying subgroups of subjects in which cluster-specific pattern of cortical reorganization may incorporate even a complex fabric of bidirectional CTh (or GI, respectively) differences in terms of both atrophy and compensational hypertrophy that might be putatively present concurrently in a subset of individuals, but not in other subgroups.

Such non-linear associations cannot be detected by methods that depend on global similarity values. With the use of dendrograms it was possible to visually inspect the pairwise similarity of the subjects in the Euclidean space and observe which clusters were sparse. The number of clusters was not determined a priori. The desirable cut off (number of clusters) was decided by using Cattell-Nelson-Gorsuch scree test (GORSUCH, R. L. Factor analysis. Lawrence Erlbaum. Hillsdale, N J, 1983).

The measure of the similarity (degree of overlap in terms of identical patient content) between clusters obtained separately from CTh and GI cluster analysis has been assessed by means of Fisher exact test.

Results

Cluster Analysis: SZ Subtyping Based on Distinct Cortical Reorganization Patterns

Data-driven hierarchical clustering analysis revealed distinct and divergent longitudinal morphometry pathways in FES patients. The analysis suggested optimal number of three clusters of patients for the data set of cortical changes within 333 parcels covering whole cortical mantle.

The clusters were named after their prevailing characteristic of GM changes patterns.

Group 1 represented severe brain atrophy.

Group 2 with brain volume expansion and ventricular shrinkage (lateral ventricular volume decrease: −8.0%/year).

Group 3 with mild brain changes

This finding suggests the existence of longitudinally defined, neuroanatomically distinct phenotypes among schizophrenia patients expressing itself in terms of distinct cortical surface remodeling during early illness stage.

Biomarkers for Schizophrenia Subtyping

According to an embodiment, the invention relates an in vitro method for diagnosing a neurodegenerative subtype of schizophrenia by determining the levels of one or more biomarkers in a sample obtained from a patient, selected from the panel consisting of S100B; NF-L; NSE; GFAP; and/or UCH-L1.

The past decade has seen efforts in the search for biomarkers for neurodegenerative diseases and traumatic brain injury. Recently, it has been recognized that some common molecular mechanisms including specific protein production are shared between almost all CNS-related disorders and brain damage that had been previously considered unrelated and biologically distinct (Lim J, Yue Z (2015) Neuronal aggregates: formation, clearance, and spreading. Dev Cell 32(4):491-501).

The inventors have found several novel circulating protein biomarkers with brain-specific origin which thus could be more suitable for assessing the subtype of schizophrenia. These developments will help clinicians to apply accessible, simple, and practical methods for early diagnosis, differential diagnosis, follow-up, and treatment assessment of schizophrenia. (Henley S M, Bates G P, Tabrizi S J (2005) Biomarkers for neurodegenerative diseases. Curr Opin Neurol 18(6):698-705).

The identification of robust blood biomarkers—that can reliably differentiate schizophrenia subtypes—could improve screening, diagnosis, and follow-up of patients with schizophrenia. The aforementioned recognition of the changes in cortical thickness and gyrification index is found to be associated with changes in a number of biomarkers, hence providing a more convenient, rapid, in vitro method of subtyping schizophrenia.

Preferred biomarkers according to the invention are S100 calcium-binding protein B (S100B), glial fibrillary acidic protein (GFAP), neurofilament-light (NF-L), Neuron-Specific Enolase (NSE) and Ubiquitin C-terminal hydrolase-L1 (UCH-L1).

S100 calcium-binding protein B (S100B) is a protein of the S-100 protein family. S100 proteins are localized in the cytoplasm and nucleus of a wide range of cells, and involved in the regulation of a number of cellular processes such as cell cycle progression and differentiation. S100 genes include at least 13 members which are located as a cluster on chromosome 1q21; however, this gene is located at 21q22.3. S100B is a calcium-binding peptide produced mainly by astrocytes. Some knowledge has been acquired from in vitro and in vivo animal experiments to understand S100B's roles in cellular energy metabolism, cytoskeleton modification, cell proliferation, and differentiation. In humans, increased S100B has been detected with various clinical conditions. Brain trauma ischemia, neurodegenerative and inflammatory diseases are associated with increased S10013 concentrations, probably due to the destruction of astrocytes. (THELIN, Eric Peter, et al. A serum protein biomarker panel improves outcome prediction in human traumatic brain injury. Journal of neurotrauma, 2019, ROTHERMUNDT, Matthias, et al. S10013 in brain damage and neurodegeneration. Microscopy research and technique, 2003, 60.6: 614-632).

In schizophrenia, serum S10013 levels may be a state marker of a limited neurodegenerative process, particularly in the early course of schizophrenia or, at least, in a subgroup of schizophrenic patients. (ALEKSOVSKA, Katina, et al. Systematic review and meta-analysis of circulating S100B blood levels in schizophrenia. PLoS One, 2014, 9.9: e106342; LARA, Diogo Rizzato, et al. Increased serum S10013 protein in schizophrenia: a study in medication-free patients. Journal of psychiatric research, 2001, 35.1: 11-14).

In addition, S10013 levels are modulated by illness duration and are related to clinical symptomatology. (SCHÜMBERG, Katharina, et al. Serum S100B is related to illness duration and clinical symptoms in schizophrenia—a meta-regression analysis. Frontiers in cellular neuroscience, 2016, 10: 46; HONG, Wu, et al. Higher plasma S100B concentrations in schizophrenia patients, and dependently associated with inflammatory markers. Scientific reports, 2016, 6: 27584).

Acute paranoid schizophrenia inpatients present a day/night change of S100B serum levels at admission that disappears at discharge. The correlation between serum S100B concentrations and the PANSS positive scores at admission as well as the decrease of S100B at discharge may be interpreted as an acute biological response to the clinical state of the patients. (MORERA-FUMERO, Armando L., et al. Day/night changes in serum S100B protein concentrations in acute paranoid schizophrenia. Progress in Neuro-Psychopharmacology and Biological Psychiatry, 2017, 75: 207-212).

Neurofilament light polypeptide (NFL), also known as neurofilament light chain, is a neurofilament protein that in humans is encoded by the NEFL gene. Neurofilament light chain is a biomarker that can be measured with immunoassays in cerebrospinal fluid and plasma and reflects axonal damage in a wide variety of neurological disorders. It is a useful marker for disease monitoring in amyotrophic lateral sclerosis, multiple sclerosis, Alzheimer's disease, and Huntington's disease.

Neuroaxonal damage and loss are the pathological substrate of many acute and chronic neurological disorders that result in permanent disability. The ability to readily detect and follow such damage would be a great advantage in the assessment of disease activity, monitoring of treatment responses and prognosis. Therefore, a biomarker that accurately reflects neuroaxonal injury would be invaluable for reaching individual therapeutic decisions and measuring drug effects in clinical trials. Until recently, measurements of the neurofilament protein that is most promising as a biomarker, neurofilament light chain (NfL), in patients with neurological disorders could only be performed with CSF samples. Currently, however, neurofilament levels in the blood can be quantified with enzyme-linked immunosorbent assay (ELISA) and more sensitive electrochemiluminescence (ECL) assay technology in many different diseases. Those highly sensitive neurofilament measurements have the potential to fill a gap in the assessment of neuroaxonal damage in various neurological disorders (multiple sclerosis, dementias, stroke, traumatic brain injury, amyotrophic lateral sclerosis, Parkinson and Huntington disease). This approach provides a sensitive assessment of the consequences of brain tissue damage with only a blood sample, an important advance to aid research and towards use of the assays in clinical practice. (ALIREZAEI, Zahra, et al. Neurofilament Light Chain as a Biomarker, and Correlation with Magnetic Resonance Imaging in Diagnosis of CNS-Related Disorders. Molecular neurobiology, 2019, 1-23; Khalil, M., Teunissen, C. E., Otto, M., Piehl, F., Sormani, M. P., Gat-tringer, T., Barro, C., Kappos, L., Comabella, M., Fazekas, F., Petzold, A., Blennow, K., Zetterberg, H., and Kuhle, J. (2018). Neurofilaments as biomarkers in neurological disorders. Nat. Rev. Neurol. 14, 577-589; Shahim, P., Gren, M., Liman, V., Andreasson, U., Norgren, N., Tegner, Y., Mattsson, N., Andreasen, N., Ost, M., Zetterberg, H., Nellgard, B., and Blennow, K. (2016). Serum neurofilament light protein predicts clinical outcome in traumatic brain injury. Sci. Rep. 6, 36791; Shahim, P., Zetterberg, H., Tegner, Y., and Blennow, K. (2017). Serum neurofilament light as a biomarker for mild traumatic brain injury in contact sports. Neurology 88, 1788-1794)

Serum NfL was proposed as a biomarker to differentiate behavioural variant frontotemporal dementia (bvFTD) from schizophrenia and bipolar disorder and to rule out neurodegeneration in the course of psychiatric disorders (AL SHWEIKI, MHD Rami, et al. Neurofilament light chain as a blood biomarker to differentiate psychiatric disorders from behavioural variant frontotemporal dementia. Journal of Psychiatric Research, 2019, 113: 137-140).

Neuron-specific enolase (NSE) is an acidic protease unique to neurons and neuroendocrine cells. It is a sensitive indicator for assessing the severity of nerve cell damage and prognosis. It is also specific markers for tumours such as neuroblastoma and small cell lung cancer (SCLC).

NSE is a glycolytic enzyme found in neuronal and neuroendocrine tissues that may play a dual role in promoting both neuroinflammation and neuroprotection in neurodegenerative events. Elevated NSE can promote extracellular matrix degradation, inflammatory glial cell proliferation, and actin remodelling, thereby affecting migration of activated macrophages and microglia to the injury site and promoting neuronal cell death. Thus, NSE could be a reliable, quantitative, and specific marker of neuronal injury. Depending on the injury, disease, and microenvironment, NSE may also show neurotrophic function as it controls neuronal survival, differentiation, and neurite regeneration. (HAQUE, Azizul, et al. New insights into the role of neuron-specific enolase in neuro-inflammation, neurodegeneration, and neuroprotection. Brain sciences, 2018, 8.2: 33).

Glial fibrillary acidic protein (GFAP) is a protein that is encoded by the GFAP gene in humans. It is a type III intermediate filament (IF) protein that is expressed by numerous cell types of the central nervous system (CNS), including astrocytes and ependymal cells during development. GFAP has also been found to be expressed in glomeruli and peritubular fibroblasts taken from rat kidneys, Leydig cells of the testis in both hamsters and humans, human keratinocytes, human osteocytes and chondrocytes and stellate cells of the pancreas and liver in rats.

Ubiquitin C-terminal hydrolase-L1 UCH-L1 is a member of a gene family whose products hydrolyse small C-terminal adducts of ubiquitin to generate the ubiquitin monomer. Expression of UCH-L1 is highly specific to neurons and to cells of the diffuse neuroendocrine system and their tumors. It is abundantly present in all neurons (accounts for 1-2% of total brain protein), expressed specifically in neurons and testis/ovary.

UCH-L1 has emerged as an important enzyme in regulating brain protein metabolism, by coupling to the proteasome pathway of protein degradation. UCH-L1 is emerging as a promising neuron-derived biomarker for traumatic brain injury, ischemic and homographic stroke, pediatric hypoxic-ischemic encephalopathy, spinal cord injury, epileptic seizure and cardiac arrest.

Interleukin-6 (IL-6) is a pleiotropic pro-inflammatory cytokine. Its deregulation is associated with chronic inflammation, and multifactorial auto-immune disorders. [48] Increasing evidence suggests a role for the involvement of immunological processes in mediating the genetic and environmental risk for schizophrenia. Indeed, schizophrenia has been associated with an abnormal activation of the immune system for many years. Previous reviews have summarized evidence linking SZ with abnormalities in various components of the immune system. In a systematic review and meta-analysis on cytokine function in medication-naive first episode psychosis a highly significant effect sizes was seen (amongst others) for IL-6 suggesting that an increase in this cytokine in first episode psychosis patients, compared with controls, is unrelated to antipsychotic drug and presumably pertains to the disease process (KAUR, Sukhvir, et al. A panoramic review of IL-6: Structure, pathophysiological roles and inhibitors. Bioorganic & medicinal chemistry, 2020, 28.5: 115327).

It is currently not completely understood how these biomarkers reach the peripheral blood. Conventional thinking is that disruption of the blood—brain barrier (BBB) results in diffusion of biomarkers from the site of injury into the general circulation. It has been shown recently that glymphatic CSF-blood exchange play a critical role in the clearance of endogenously produced proteins, in this case biomarkers of brain damage, from the CNS to the peripheral blood. This approach therefore allows for very early detection of even subtle brain abnormalities, such as those changes in cortical thickness and gyrification index described above, independent upon disrupted BBB, that otherwise pertains to the later stages of schizophrenia.

A data-driven method to identify latent classes of first-episode schizophrenia spectrum patients (FES) based on distinct patterns of progressive brain reorganization in subjects recruited during early illness stage with previously evidenced steepest gradient of neuroprogression.

Methods: FES and matching healthy controls completed two high-resolution T1-weighted structural MRI scans at 1 year interval. Euclidean hierarchical cluster analysis was applied to the data of longitudinal change in both cortical thickness and cortical gyrification extracted from cortical parcels.

Both cortical thickness and gyrification data-driven models revealed independently albeit with statistically significant overlap (Fisher's exact, p=0.001) bidirectional longitudinal morphometry pathways in FES: Cluster 1 displayed neurodegenerative changes with mild overall change in cortical thickness. Cluster showed rapid cortical atrophy; Cluster 3 showed GM volume increase along with substantial ventricular shrinkage.

Conclusions: This study suggests divergent and clinically relevant features of brain reorganization in schizophrenia.

All citations mentioned herein are incorporated by reference.

1. A method for determining a subtype of schizophrenia in a subject, said method comprising:
a. obtaining a first structural magnetic resonance imaging (sMRI) brain scan at a first point in time;
b. obtaining a second structural magnetic resonance imaging brain scan at a later point in time;
c. processing the first and second scans to obtain first and second measures of cortical thickness and/or a gyrification index;
d. comparing the first and second measures of cortical thickness to establish the change in cortical thickness and/or gyrification index;
e. clustering the subject as belonging to distinct subtypes of schizophrenia according to the changes in cortical thickness and/or gyrification index.

2. The method according to claim 1 wherein the first and second structural magnetic resonance imaging brain scans are taken at least six months apart, preferably at least one year apart.

3. The method according to claim 1, wherein the first structural magnetic resonance imaging (sMRI) brain scan is taken soon after the onset of the symptoms of schizophrenia.

4. The method according to claim 1, wherein the processing step c comprises applying an atlas of cortical parcellation based on a resting-state functional connectivity boundary maps to multiple contiguous areas of the subject's cortical surface at both the time points.

5. The method according to claim 1, wherein the distinct subtypes are associated with i) substantially no change in mean cortical thickness; ii) a reduction in cortical thickness; and iii) an increase in cortical thickness.

6. A method according to claim 1, wherein the subtypes are characterised by,
a. region-specific local neurodegenerative changes without gross change in overall cortical thickness;
b. widespread cortical atrophy;
c. cortical hypertrophy with ventricular shrinkage.

7. A method according to claim 1, wherein the subject is a first-episode schizophrenia spectrum (FES) patient.

8. An in vitro method for determining a subtype of schizophrenia in a subject, the method comprising determining in a biological sample of a subject the level of at least one biomarker selected from the group consisting of S100 calcium-binding protein B (S100B), Neurofilament-light (NF-L), Neuron-Specific Enolase (NSE), Glial fibrillary acidic protein (GFAP), and Ubiquitin C-terminal hydrolase-L1 (UCH-L1) and interleukin 6 (Il-6).

9. A method according to claim 8, wherein the level of at least two biomarkers, such as two, three or four biomarkers, is determined.

10. The method according to claim 8, wherein the biomarkers are selected from S100 calcium-binding protein B (S100B), Neurofilament-light (NF-L), interleukin 6 (Il-6) and Neuron-Specific Enolase (NSE).

11. A method according to claim 8 wherein the subtypes are characterised by,
a. region-specific local neurodegenerative changes confined to the ventral attention network without gross change in overall cortical thickness;
b. widespread cortical atrophy: or
c. cortical hypertrophy with ventricular shrinkage.

12. A method of treatment of schizophrenia in a patient, comprising the steps of:
i. determining the subtype of schizophrenia in the patient by a method, said method comprising:
a. obtaining a first structural magnetic resonance imaging (sMRI) brain scan at a first point in time;
b. obtaining a second structural magnetic resonance imaging brain scan at a later point in time;
c. processing the first and second scans to obtain first and second measures of cortical thickness and/or a gyrification index;
d. comparing the first and second measures to establish the change in cortical thickness and/or gyrification index;
e. clustering the subject as belonging to distinct subtypes of schizophrenia according to the changes in cortical thickness and/or gyrification index;
ii. selecting an antipsychotic medication based on the determination in step i; and
iii. administering the antipsychotic medication to the patient.

13. (canceled)

14. The method of claim 12 wherein the medicament is selected from chlorpromazine, fluphenazine, haloperidol, perphenazine, thioridazine, thiothixene, trifluoperazine, aripiprazole, aripiprazole lauroxil, asenapine, brexpiprazole, cariprazine, clozapine, iloperidone, lumateperone tosylate, lurasidone, olanzapine, paliperidone, paliperidone palmitate, quetiapine, risperidone, and ziprasidone.

15. A method of determining the efficacy of an antipsychotic medication in a patient suffering from schizophrenia, comprising the steps of:
i. determining the subtype of schizophrenia in the patient by a method, said method comprising:
a. obtaining a first structural magnetic resonance imaging (sMRI) brain scan at a first point in time;
b. obtaining a second structural magnetic resonance imaging brain scan at a later point in time;
c. processing the first and second scans to obtain first and second measures of cortical thickness and/or a gyrification index;
d. comparing the first and second measures to establish the change in cortical thickness and/or gyrification index;
e. clustering the subject as belonging to distinct subtypes of schizophrenia according to the changes in cortical thickness and/or gyrification index;
ii. administering the medication to the patient; and
iii. assessing the efficacy of the medication in reducing the symptoms of schizophrenia.

16. A method of determining the efficacy of antipsychotic medication to patients suffering from a subtype of schizophrenia, comprising the steps of:
i. providing a cohort of patients suffering from schizophrenia;
ii. determining the subtype of schizophrenia patient by a method, said method comprising:
a. obtaining a first structural magnetic resonance imaging (sMRI) brain scan at a first point in time;
b. obtaining a second structural magnetic resonance imaging brain scan at a later point in time;
c. processing the first and second scans to obtain first and second measures of cortical thickness and/or a gyrification index;
d. comparing the first and second measures to establish the change in cortical thickness and/or gyrification index;
e. clustering the subject as belonging to distinct subtypes of schizophrenia according to the changes in cortical thickness and/or gyrification index;
iii. administering the medication to the patients; and
iv. assessing the efficacy of the medication in alleviating the symptoms of schizophrenia in each subtype.
